(19) United States
(12) Patent Application Publication
(10) Pub. No.: US 2013/0041379 A1
(43) Pub. Date: Feb. 14, 2013

(54) Surgical Rongeur Release Mechanism

(71) Applicants: Peter Pal Bodor, Pembroke Pines, FL (US); Shusheng Ye, Davie, FL (US); Leighton Schonlau, Weston, FL (US); Zoltan A. Bodor, Plantation, FL (US); David Bush, Pembroke Pines, FL (US)

(72) Inventors: Peter Pal Bodor, Pembroke Pines, FL (US); Shusheng Ye, Davie, FL (US); Leighton Schonlau, Weston, FL (US); Zoltan A. Bodor, Plantation, FL (US); David Bush, Pembroke Pines, FL (US)

(73) Assignee: Integrated Medical Systems International, Inc., Birmingham, AL (US)

(21) Appl. No.: 13/587,472

(22) Filed: Aug. 16, 2012

Related U.S. Application Data
(63) Continuation-in-part of application No. 13/083,742, filed on Apr. 11, 2011.
(60) Provisional application No. 61/524,452, filed on Aug. 17, 2011.
(60) Provisional application No. 61/523,952, filed on Aug. 16, 2011.

(51) Int. Cl. A61B 17/16

(52) U.S. Cl. 606/83

(57) ABSTRACT

A rongeur including a shaft having a foot plate, a cutting slide, a breach extending between the foot plate and the cutting slide and a handle assembly for moving the cutting slide along the shaft. The cutting slide includes a pair of tracking arms arranged about a distal end of the cutting slide and a third tracking arm arranged about the proximal end thereof. The tracking arms are received within corresponding tracking slots in the shaft when the rongeur is in a closed position. The tracking arms are disengaged from the tracking slots by rotating the cutting slide about a pivot axis that extends parallel to the length of the shaft thereby placing the rongeur in an open position. To prevent inadvertent disengagement of the tracking arms from the tracking slots, a trigger assembly is provided which arrests proximal movement of the cutting slide.

### CROSS-REFERENCES TO RELATED APPLICATIONS

This application is a continuation in part of and claims priority to U.S. provisional application Ser. No. 61/524,452 filed on Aug. 17, 2011, U.S. provisional application Ser. No. 61/523,952 filed on Aug. 16, 2011, and U.S. non-provisional application Ser. No. 13/083,742 filed on Apr. 11, 2011, which claims priority to U.S. provisional application Ser. No. 61/438,754 filed on Feb. 2, 2011, the disclosures of which are incorporated herein by reference in their entireties for any and all purposes.

### FIELD OF THE INVENTION

This invention relates generally to surgical rongeurs, and more particularly to release mechanisms for Kerrison-style rongeurs used in laminectomies and laminotomies.

### BACKGROUND OF THE INVENTION

Kerrison rongeurs are utilized in spinal surgery to remove bone and tissue and thereby gain access to the spinal canal. Kerrison rongeurs typically comprise a stationary shaft and a cutting slide that is longitudinally slidable relative to the stationary shaft. At the distal end of the cutting slide is a cutting edge which engages a foot plate that is located at the distal end of the stationary shaft. The cutting edge on the cutting slide and the foot plate on the stationary shaft are commonly referred to as the “cutting jaws”. When a Kerrison rongeur is in use, the surgeon places the bone to be cut, such as the leading edge of the lamina of a vertebrae, within the cutting jaws. The surgeon then squeezes the handle of the rongeur which causes the cutting edge of the cutting slide to be advanced through that portion of bone to reach the foot plate and thereby amputating that portion of bone.

During use of a Kerrison rongeur, blood, tissue and bone can accumulate between the stationary shaft and cutting slide. Since the accumulated materials are often difficult to remove, subsequent uses of an improperly cleaned rongeur can result in cross-contamination events. For this reason, attempts have been made to design a Kerrison rongeur that can be quickly and easily, fully or partially disassembled to provide access to the spaces between the stationary shaft and cutting slide where the accumulated materials reside.

U.S. Pat. No. 6,126,674 to Janzen describes a Kerrison rongeur that can be completely disassembled by means of a pin that can slide out over a slot in a trigger thereby releasing the slide. A drawback of the device is that the parts can become lost during cleaning and sterilization. U.S. Pat. No. 6,723,103 to Edwards describes a Kerrison rongeur that when held in a retracted position by means of an elastic member, a gap is formed between the cutting slide and the stationary shaft to provide access there between. However, this gap is not sufficiently wide to allow unfettered access between the slide and shaft by brushes for thorough cleaning. Further, the distal portions of the cutting side and stationary shaft remain in contact where the accumulated materials cannot be easily cleaned out. U.S. Pat. No. 5,961,531 to Weber and German Patent Application No. DE102009006689 to Heinemann each describe a convertible rongeur that opens up in an alligator-jaw-like manner which exposes the surfaces between the cutting slide and the stationary shaft. Although the converted devices remain assembled in a single piece since the cutting slides are left dangling in an open position, the devices tend to occupy more space in the sterilization chamber than they do in the closed position and increase the likelihood of the cutting slide becoming bent or otherwise damaged.

U.S. Pat. No. 6,802,852 to Tontarra describes a rongeur employing a spring loaded pivoting device that limits the rotation of the trigger. To release the movable blade, the trigger is first squeezed with one hand and the locking device is pivoted up and away from the path of the trigger and hammer by the thumb of the same hand. The trigger is then released allowing the blade to move into its back position where it can be lifted up or completely removed from the handle for cleaning. U.S. Pat. No. 7,052,505 to Widmann describes a rongeur employing a spring loaded sliding element for limiting the rearward movement of the blade. Depressing the sliding element allows the blade to move into its back position and become separated from the main handle.

### SUMMARY OF THE INVENTION

The present invention is directed to a convertible Kerrison rongeur that can be partially disassembled for exposing the sliding surfaces of the rongeur for thorough cleaning while remaining compact and as a single piece. This is accomplished by providing the rongeur with a hinging element having a pivot axis that extends parallel to the functional length of the rongeur. Such an arrangement allows a top cutting slide of the rongeur to flip between an open position where the cutting slide is located off to one side of the rongeur and a closed position.

According to one aspect of the invention, there is provided a rongeur including a shaft member having a length that terminates in a foot plate, a cutting slide slideably coupled with the shaft member and terminating in a cutting edge and means for slidably moving the cutting slide longitudinally relative to the shaft member. To slide cutting slide relative to the shaft member, the cutting slide is provided with a pair of tracking arms arranged about a distal end of cutting slide and a third tracking arm arranged about the proximal end of the cutting slide. These tracking arms are slidably and detachably received within corresponding tracking slots in the shaft member. A hinge assembly is positioned between the cutting slide and the shaft member that has a pivot axis that extends substantially parallel to the length of the shaft member. By combining the sliding and pivoting functions, the cutting slide is allowed to slide longitudinally with respect to the shaft member, and when desired, the tracking arms disengaged from the tracking slots and the cutting slide pivoted off to the side of the shaft to expose the inner, sliding surfaces of the cutting slide and the shaft member for cleaning and repairs.

According to another aspect of the invention there is provided a surgical rongeur including a bottom shaft having a length terminating in a foot plate, a top shaft slidably coupled to the bottom shaft and terminating in a cutting edge, a breach formed between the cutting edge and the foot plate, a handle assembly configured for selectively sliding the top shaft along the bottom shaft, and a pivot axis extending substantially parallel to the length of the bottom shaft. The pivot axis runs through a pin that is fixed to the bottom shaft and slideably and rotatably received by the top shaft. This allows the top shaft to rotate about the pin thereby exposing the inner sliding surfaces of the bottom shaft and the top shaft. In one embodiment, the pin extends through an opening in a sliding member of the top shaft that is slidably received within a gap in the bottom shaft. The opening is elongated radially relative to the pivot axis thus allowing the top shaft to shift radially upward and away from the bottom shaft which is required for disengaging the top shaft from the bottom shaft before rotating the top shaft about the pin.

According to yet another aspect of the invention, there is provided a surgical rongeur including a bottom shaft having a foot plate, a top shaft having a cutting edge, a breach extending between the cutting edge and the foot plate for receiving a human tissue to be cut, a tracking assembly for slidably coupling the bottom shaft with the top shaft, the tracking assembly including at least one tracking arm and at least one tracking slot and a pivot assembly for pivotably coupling the bottom shaft with the top shaft, the pivot assembly having a pivot axis that extends substantially parallel to a length of the bottom shaft. The pivot assembly and the tracking assembly are arranged to selectively transform the rongeur between an open position and a closed position. When the rongeur is in the closed position, the at least one tracking arm is slidably engaged with the at least one tracking slot and the rongeur in ready for use. When the rongeur is in the open position, the at least one tracking arm is disengaged from the at least one tracking slot and the top shaft to rotated about the pivot axis to expose the inner sliding surfaces of the top and bottom shafts.

To prevent inadvertent disengagement of the tracking arms from the tracking slots and thus adjustment of a rongeur between opened and closed positions, a trigger assembly is provided which selectively arrests proximal movement of the cutting slide along the shaft. According to this aspect of the invention, there is provided a surgical rongeur including a shaft member having a foot plate, a cutting slide having a cutting edge that is engagable with the foot plate, and a trigger pivotably coupled to the shaft member. The trigger includes a neck portion that extends through the shaft member and a hammer portion that is received within a niche in the cutting slide. The shaft member includes a pathway along which the neck is configured to travel when the trigger is depressed and extended, the pathway including a section having a variable width that is selectively varied between a first width that is less than a width of the neck and a second width that is greater than or equal to the width of the neck. The variable width can be varied by arranging within the section a first flexible strip to a first side of the neck and a second flexible strip to a second side of the neck, each of the first flexible strip and the second flexible strip including a bend. Alternatively, the variable width can be varied by including within the section a pair of bearings that are biased against opposing sides of the section.

According to another aspect of the invention, there is provided a surgical rongeur including a bottom shaft having a foot plate, a top shaft slidably coupled to the bottom shaft, the top shaft having a cutting edge, and a trigger assembly. The trigger assembly includes a trigger having a neck portion that extends through a pathway in the bottom shaft and an upper end that is removably engaged with the top shaft. The pathway includes a section having an adjustable width that is adjustable between a first width that is less than a width of the neck and a second width that is greater than or equal to the width of the neck. The trigger assembly may include a spring-biased member that extends into a proximal end of the pathway for prevent over-pivoting of the trigger neck proximally within the pathway.

According to yet another aspect of the invention, there is provided a surgical rongeur including a bottom shaft having a foot plate, a top shaft having a cutting edge, a breach formed between the foot plate and the cutting edge, a tracking assembly slidably coupling the bottom shaft with the top shaft, and a trigger assembly having a trigger with a neck portion that extends into a pathway in the bottom shaft and an upper end that extends into an opening in the top shaft, the pathway including a section having an expandable width. The expandable width includes a first width that is less than a width of the neck and a second width that is equal to or greater than the width of the neck and may be defined between flexible metal pieces or spring-biased members. The handle assembly includes a finger-actuated blocking member having a rigid portion that extends into a proximal end of the pathway and a flexible portion that extends into a handle portion and bears against the rigid portion.

### BRIEF DESCRIPTION OF THE DRAWINGS

FIG. 1 is perspective view of a rongeur in accordance with a preferred embodiment of the present invention.

FIG. 2 is an exploded view of the rongeur of FIG. 1.

FIG. 3 is a perspective view of the rongeur of FIG. 1 illustrating a cutting edge of the cutting slide engaged with a foot plate of the stationary shaft.

FIG. 4 is a perspective view of the rongeur of FIG. 1 illustrating the alignment of tracking arms of the cutting slide with disengaging sections of corresponding tracking slots of the stationary shaft.

FIG. 5 is a perspective view of the rongeur of FIG. 1 illustrating the disengagement of the tracking arms from the corresponding tracking slots and pivoting of the cutting slide.

FIG. 6 is a perspective view of the rongeur of FIG. 1 illustrating further pivoting of the cutting slide.

FIG. 7 is a sectional view of the rongeur of FIG. 1 illustrating a closed position.

FIG. 8 is a sectional view of the rongeur of FIG. 1 illustrating an open position.

FIG. 9 is an exploded view of a rongeur in accordance with another preferred embodiment of the present invention.

FIG. 10 is a perspective view of the rongeur of FIG. 9 illustrating the disengagement of tracking arms of the cutting slide from corresponding tracking slots of the stationary shaft.

FIG. 11 is a perspective view of the rongeur of FIG. 9 illustrating pivoting of the cutting slide.

FIG. 12 is a sectional view through a pivot assembly of the rongeur of FIG. 9 illustrating a closed position.

FIG. 13 is a sectional view of the rongeur of FIG. 14.

FIG. 14 is a sectional view of the rongeur of FIG. 9 illustrating an open position.

FIG. 15 is an exploded view of a rongeur displaying a release mechanism in accordance with a preferred embodiment of the present invention.

FIG. 16 is a perspective view of the rongeur of FIG. 15 arranged in a neutral position.

FIG. 17 is a partial sectional view of the rongeur of FIG. 16 illustrating the release mechanism in a locked position.

FIG. 18 is a perspective view of the rongeur of FIG. 15 arranged in an unlocked position.

FIG. 19 is a partial sectional view of the rongeur of FIG. 18 illustrating the release mechanism when the rongeur is in an unlocked position.

FIG. 20 is a perspective view of the rongeur of FIG. 15 arranged in an opened position.

FIG. 21 is perspective view of release mechanism of FIG. 15 in a locked position.

FIG. 22 is a sectional view of the rongeur of FIG. 15 illustrating the release mechanism when the rongeur is in a neutral position.

FIG. 23 is a sectional view of the rongeur of FIG. 15 illustrating the release mechanism when the rongeur is in an unlocked position.

FIG. 24 is perspective view of a rongeur release mechanism in accordance with a preferred embodiment of the present invention in a locked position.

FIG. 25 is perspective view of the rongeur release mechanism of FIG. 24 in an unlocked position.

FIG. 26 is a sectional view of the rongeur release mechanism of FIG. 24 in a locked position.

FIG. 27 is a sectional view of the rongeur release mechanism of FIG. 24 in an unlocked position.

FIG. 28 is a perspective view of a trigger of the rongeur of FIG. 24.

FIG. 29 is a sectional view of a trigger neck of the rongeur of FIG. 24.

FIG. 30 is perspective view of a rongeur release mechanism in accordance with a preferred embodiment of the present invention in a locked position.

FIG. 31 is partial sectional view of the rongeur release mechanism of FIG. 30 in a locked position.

FIG. 32 is partial sectional view of the rongeur release mechanism of FIG. 30 in an unlocked position.

FIG. 33 is partial sectional view of the rongeur release mechanism of FIG. 30 in an opened position.

### DETAILED DESCRIPTION OF PRESENTLY PREFERRED EMBODIMENTS

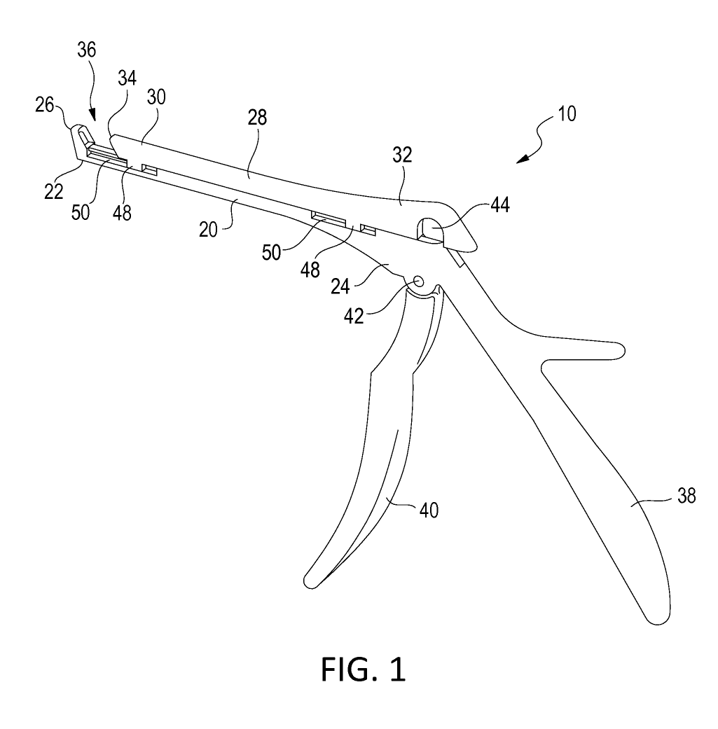

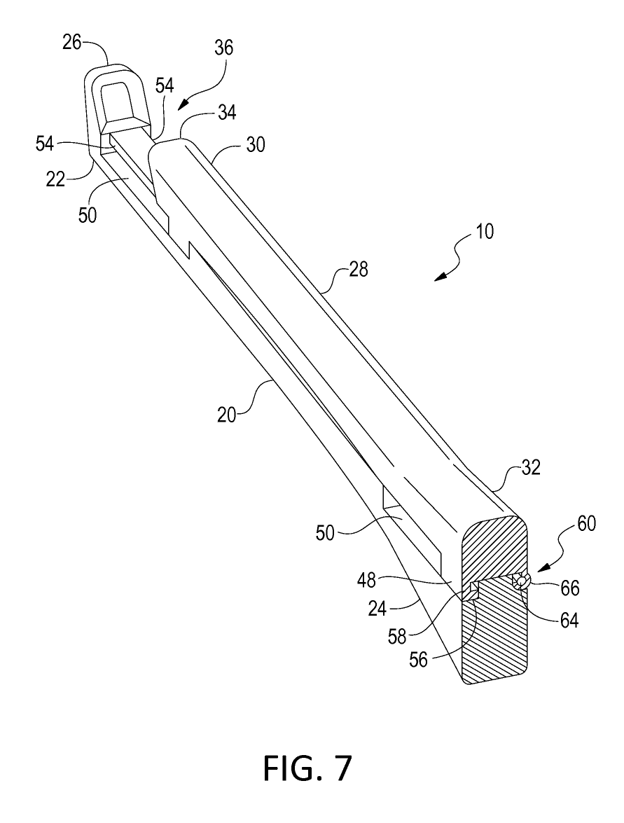

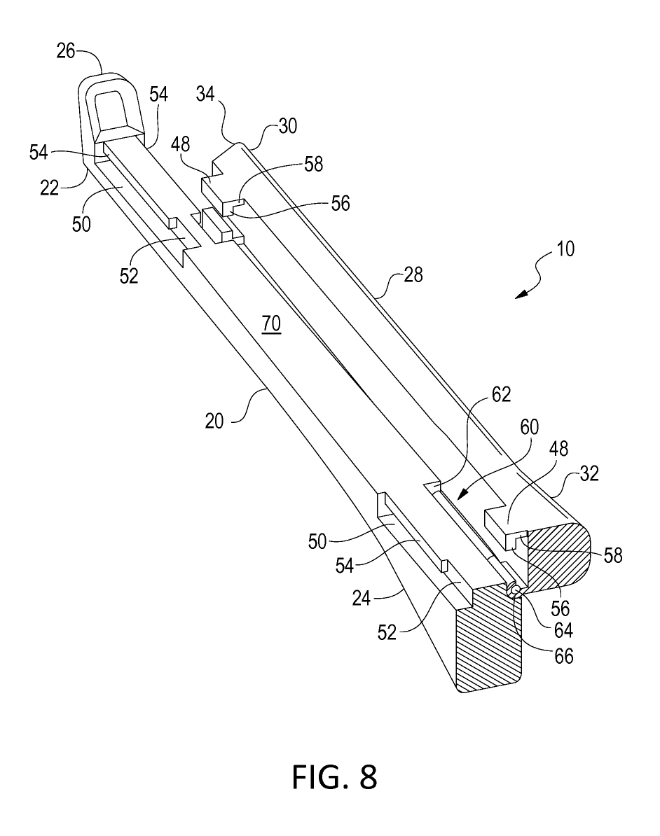

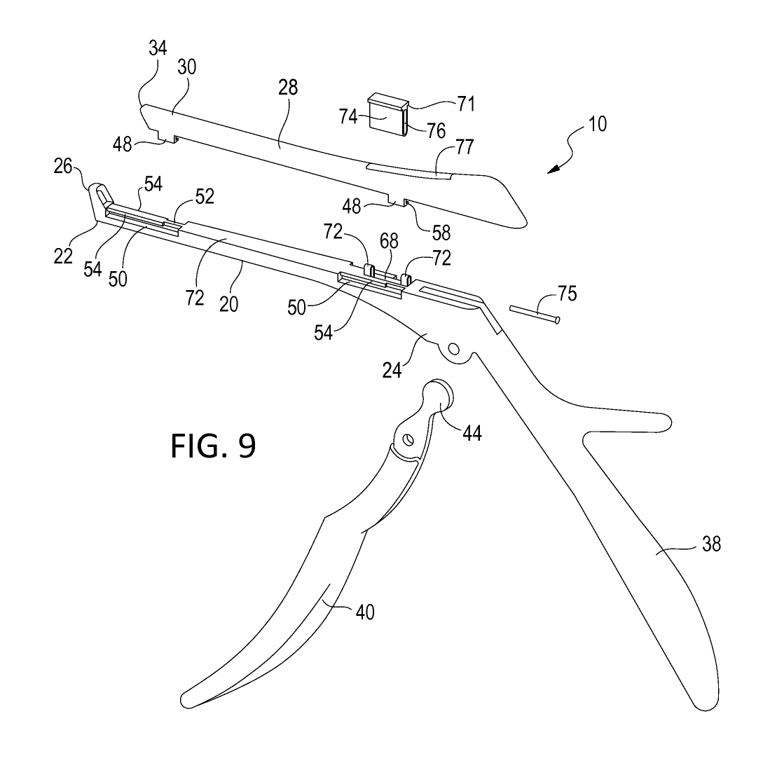

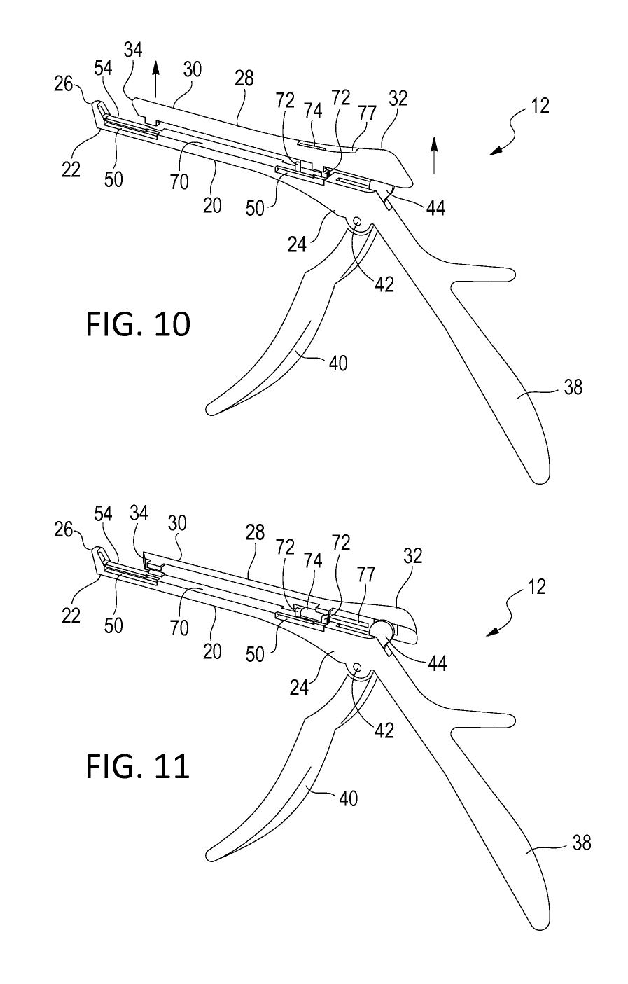

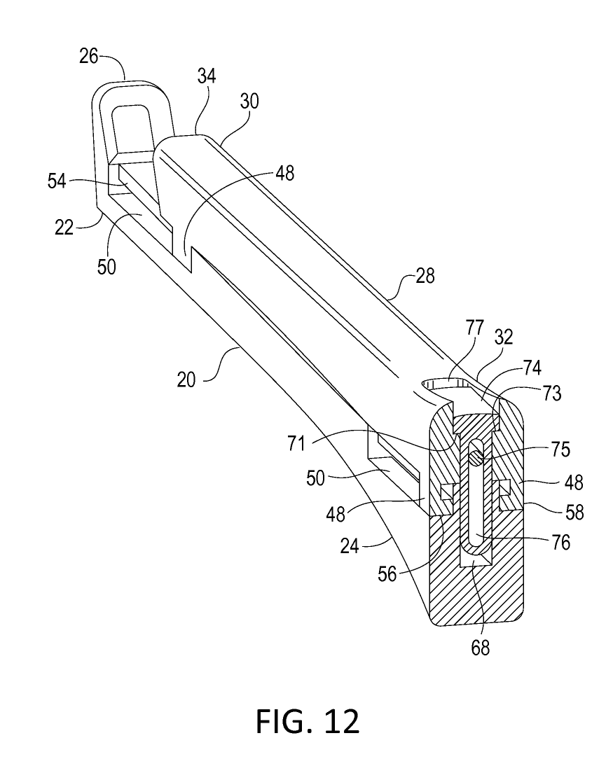

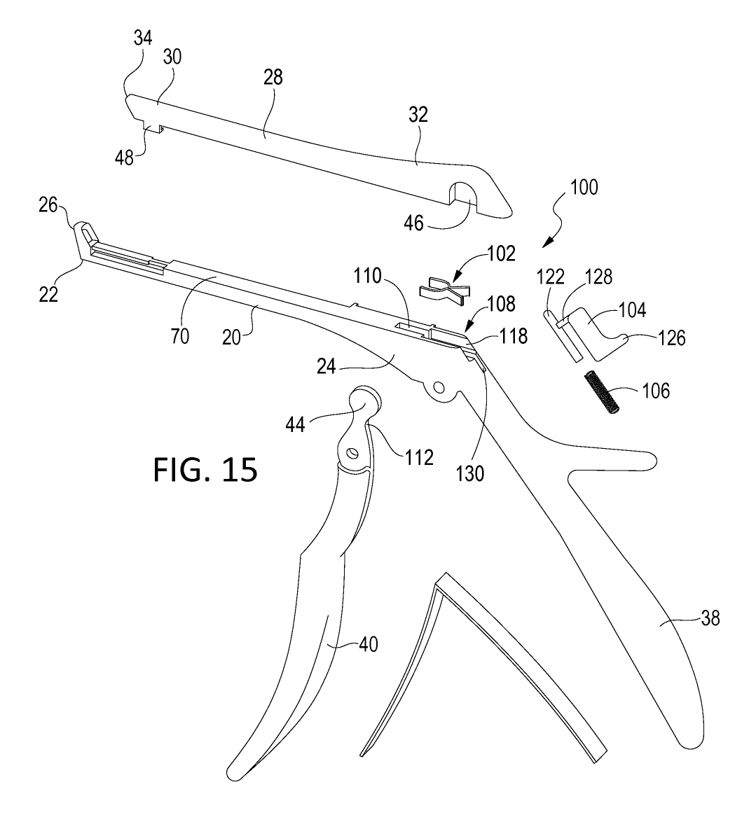

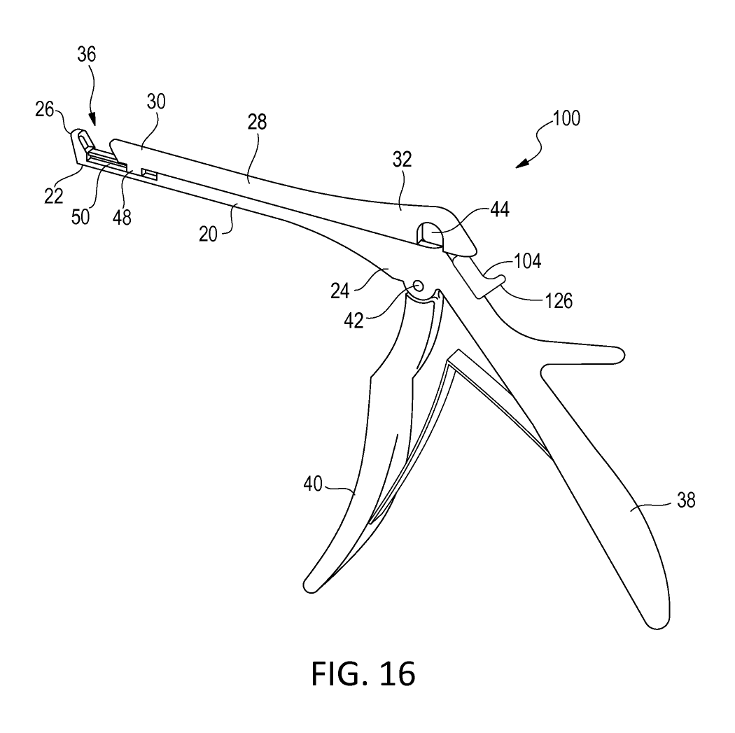

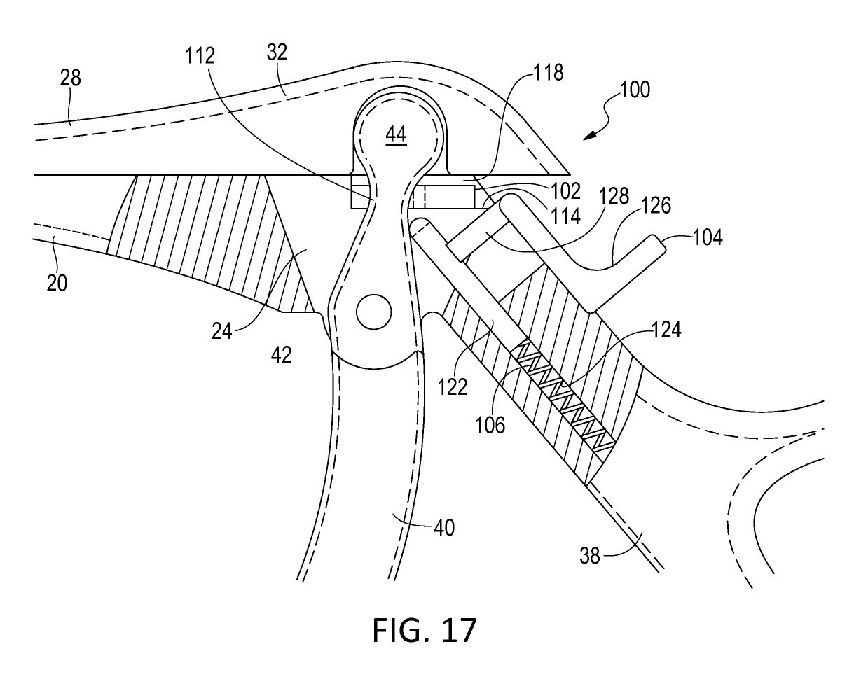

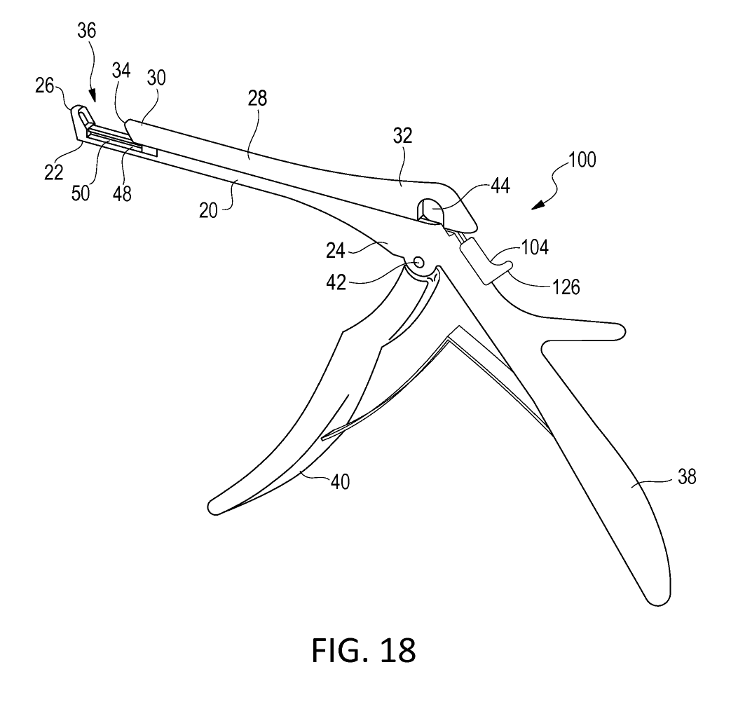

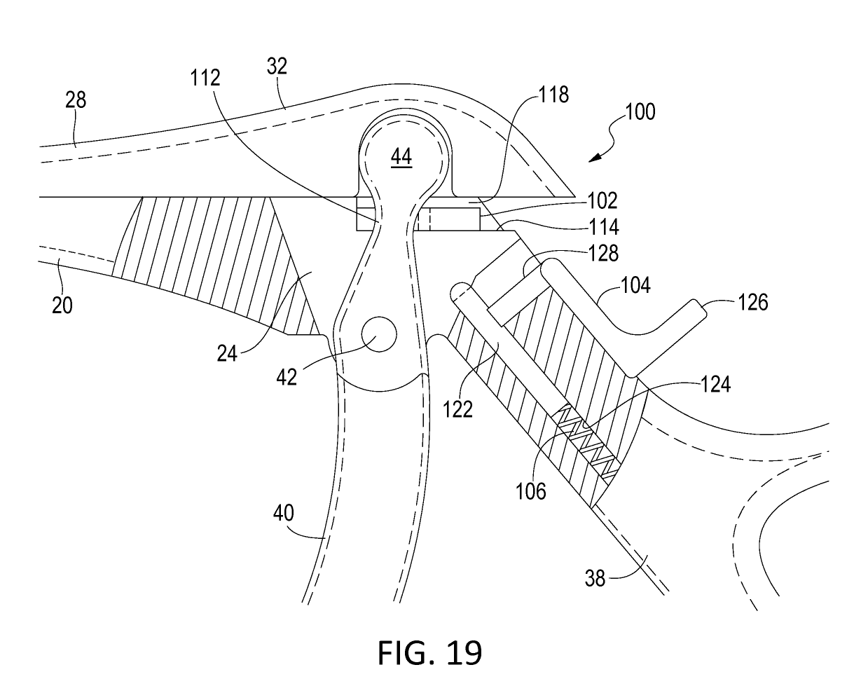

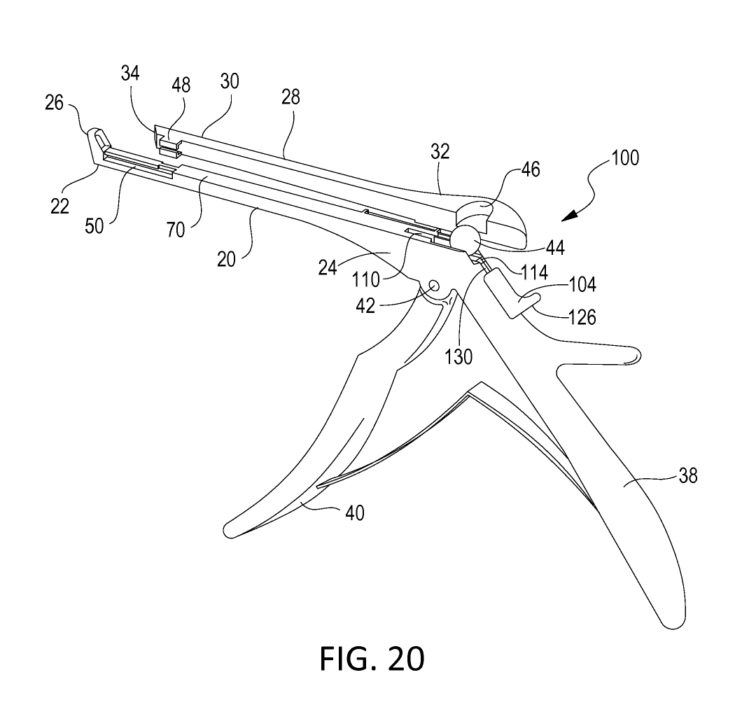

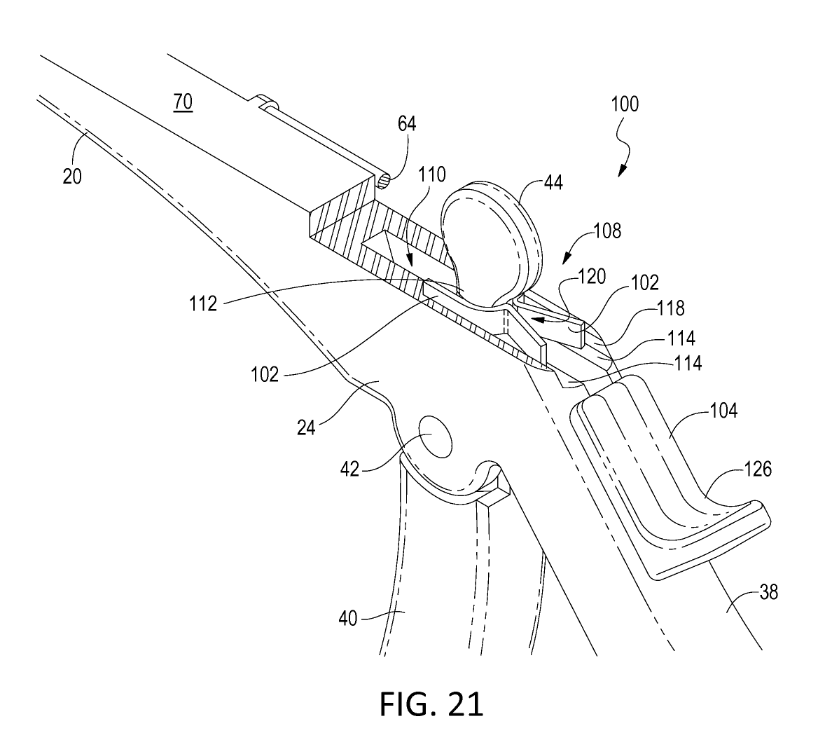

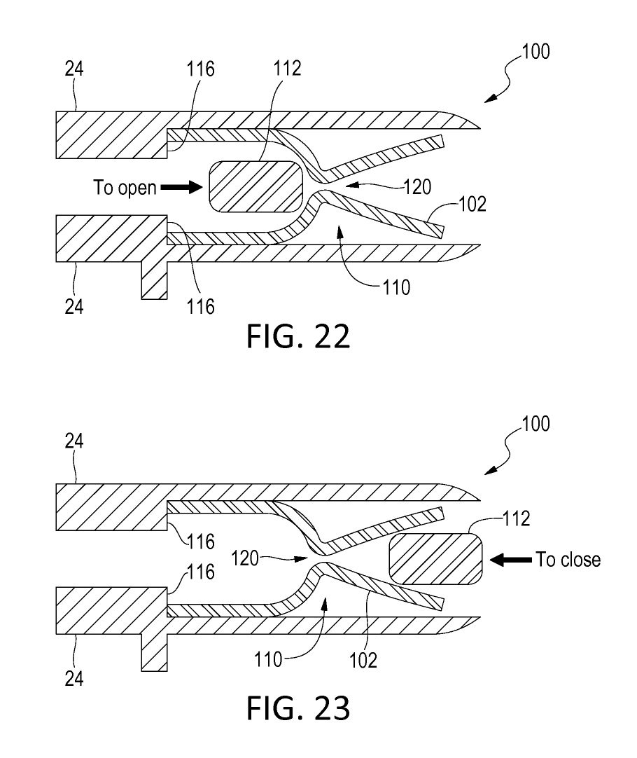

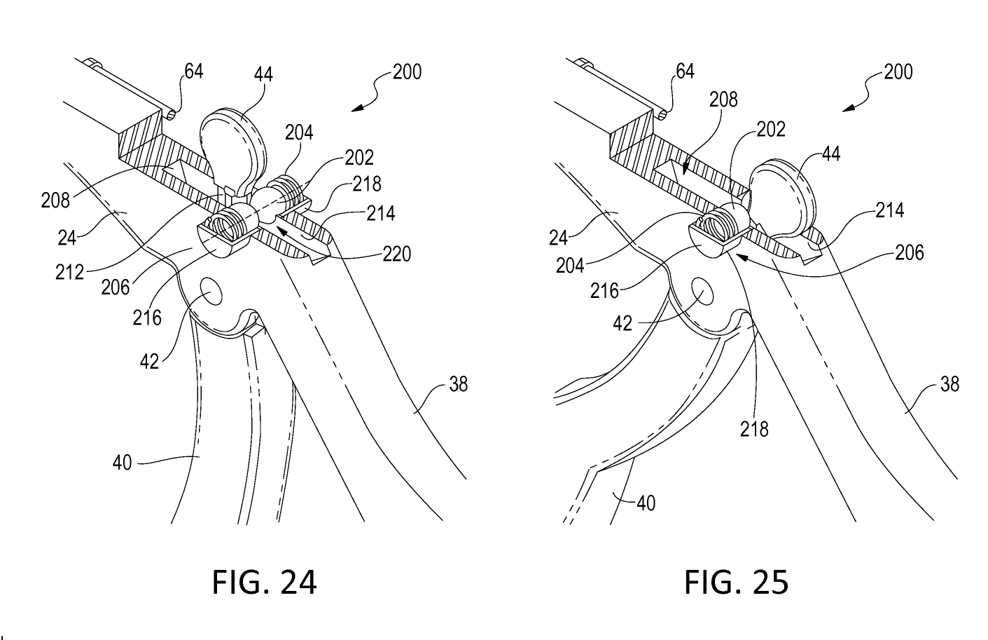

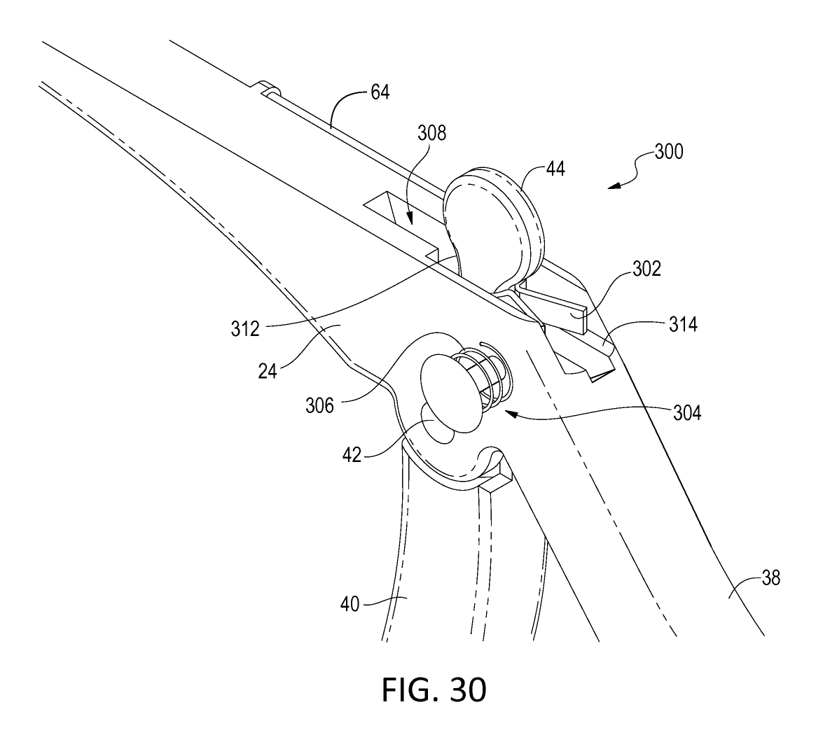

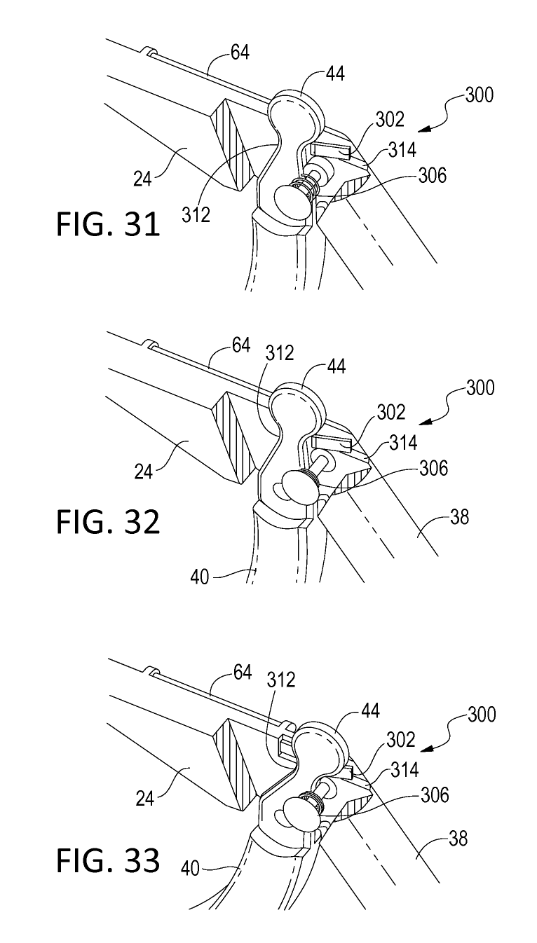

FIGS. 1 through 8 illustrate a surgical rongeur 10 in accordance with a preferred embodiment of the present invention. A rongeur 12 in accordance with another preferred embodiment of the present invention is illustrated in FIGS. 9 through 14, where like features share like numbering with FIGS. 1 through 8. FIGS. 15 through 23 illustrate a rongeur release mechanism 100 in accordance with a preferred embodiment of the present invention. FIGS. 24 through 28 illustrate a rongeur release mechanism 200 in accordance with another preferred embodiment of the present invention. A rongeur release mechanism 300 in accordance with yet another preferred embodiment of the present invention is illustrated in FIGS. 30 through 33. In FIGS. 15 through 33, like features share like numbering with FIGS. 1 through 8.

Referring to FIGS. 1 through 14, each of rongeurs 10 and 12 generally includes a stationary shaft 20 having a distal end 22, a proximal end 24 and a foot plate 26. A cutting slide 28 having a distal end 30 and a proximal end 32 is slidably coupled with and partially detachable from a top side of shaft 20. Distal end 30 of cutting slide 28 terminates in a cutting edge 34 that together with foot plate 26 defines a breach 36 for receiving bone or other tissue to be cutt.

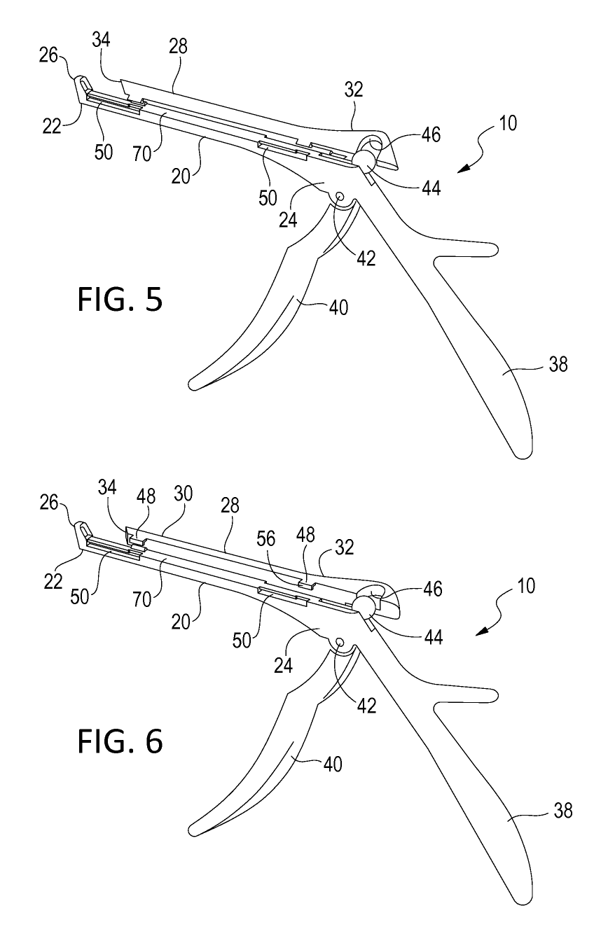

To cut tissue received within breach 36, cutting edge 34 is pressed against foot plate 26 by compressing a handle assembly including a stationary first handle 38 that is integral with proximal end 32 of cutting slide 28 and a second handle 40 that is pivotably coupled to first handle 38 at point 42. At the top of second handle 40 is a head 44 that is received within an open notch 46 (only in rongeur 10) in proximal end 32 of cutting slide 28. As best illustrated in FIG. 5, when the handle assembly is compressed, second handle 40 is pivoted proximally toward first handle 38 causing head 44 to pivot distally toward foot plate 26, in turn causing cutting slide 28 to slide distally until cutting edge 34 engages foot plate 26.

Liker prior art rongeurs, when each of rongeurs 10 and 12 is used in surgical procedures, blood, bone and other tissue can accumulate between shaft 20 and cutting slide 28. As described below in further detail, to facilitate cleaning between shaft 20 and cuttings slide 28, each of rongeurs 10 and 20 is configured to partially detach cutting slide 28 from shaft 20 and pivot cutting slide 28 off to a lateral side of shaft 20. This is accomplished by providing each of rongeurs 10 and 12 with a hinge assembly having a pivot axis that extends substantially parallel with the functional lengths of cutting slide 28 and shaft 20 and a tracking assembly that enables sliding shaft 10 to remain slidably coupled to shaft 20 during cutting operations and partially de-coupled from shaft 20 when cleaning is required.

Referring to FIG. 1, there is shown rongeur 10 in a neutral position. In the neutral position, handle 40 is held in place by a spring mechanism (not shown) with handle 40 being neither depressed nor extended. In this position, the tracking assembly is arranged to maintain cutting slide 28 fully engaged with shaft 20 by slidably seating a plurality of L-shaped tracking arms 48, which extend downwardly from cutting slide 28, within corresponding L-shape tracking slots 50 formed within shaft 20. This arrangement prevents any substantial lateral or vertical movement between cutting slide 28 and shaft 20.

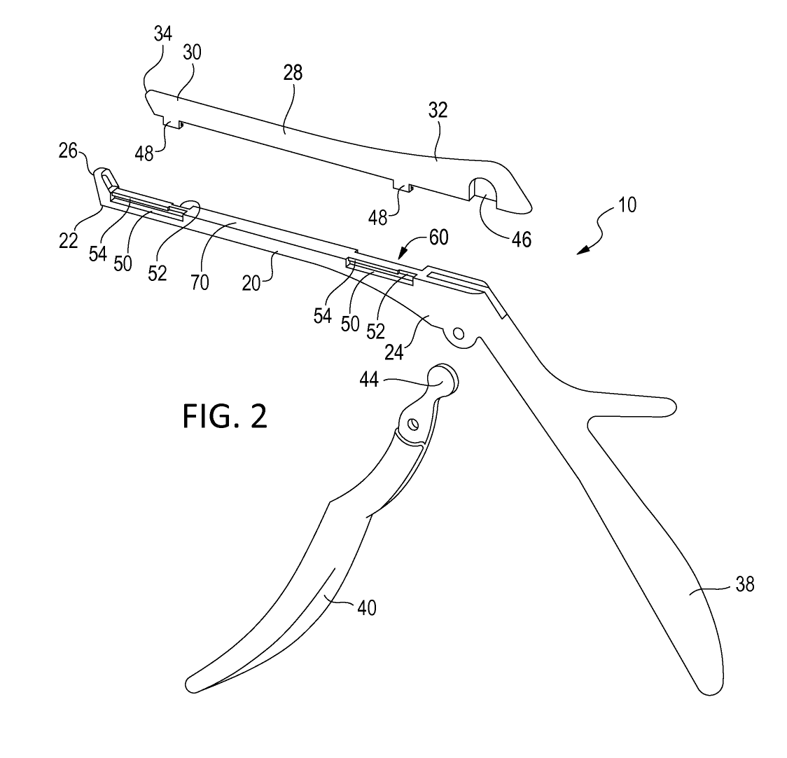

More particularly, referring to FIGS. 2, 7 and 8, the tracking assembly of rongeur 12 includes a pair of opposing tracking slots 50 that are formed in distal end 22 on the lateral sides of shaft 20 immediately proximal to foot plate 22. A third tracking slot 50 is formed within proximal end 24 of shaft 20 on a lateral side thereof opposite the hinge assembly. Each tracking slot 50 extends longitudinally along shaft 10 and is open to a lateral side of shaft 10. Each slot 50 has a smooth lower surface, vertically extending end walls defining the length of slot 50 and an interior wall 52 extending vertically from the lower surface and longitudinally between the end walls. Atop each slot 50 and supported by interior wall 52 is an overhang 54. Each overhang 54 extends laterally from the interior wall 52 above the lower surface of slot 50 but stops short of extending to the plane formed by a lateral side of shaft 20. This creates an L-shaped space having a vertical portion that is that defined between the exposed edge and upper surface of overhang 54 and the plane formed by the lateral side of shaft 20 and a horizontal portion defined by a lower surface of overhang 54, the lower surface of slot 50, interior wall 52 and the plane formed by the exposed edge of overhang 54. The L-shaped space of each slot 50 is configured for receiving a respective L-shaped tracking arm 38 of cutting slide 28 with a horizontal portion 56 of the tracking arm 38 being slidably engaged with the lower surface and interior wall 52 of the slot 50 and the lower surface of overhang 54 and a vertical portion 58 of the tracking arm 38 being slideably engaged with the exposed lateral edge of overhang 54. Arranged in this manner, each tracking arm 48 is locked within a respective tracking slot 50 and allowed to slide longitudinally along shaft 20.

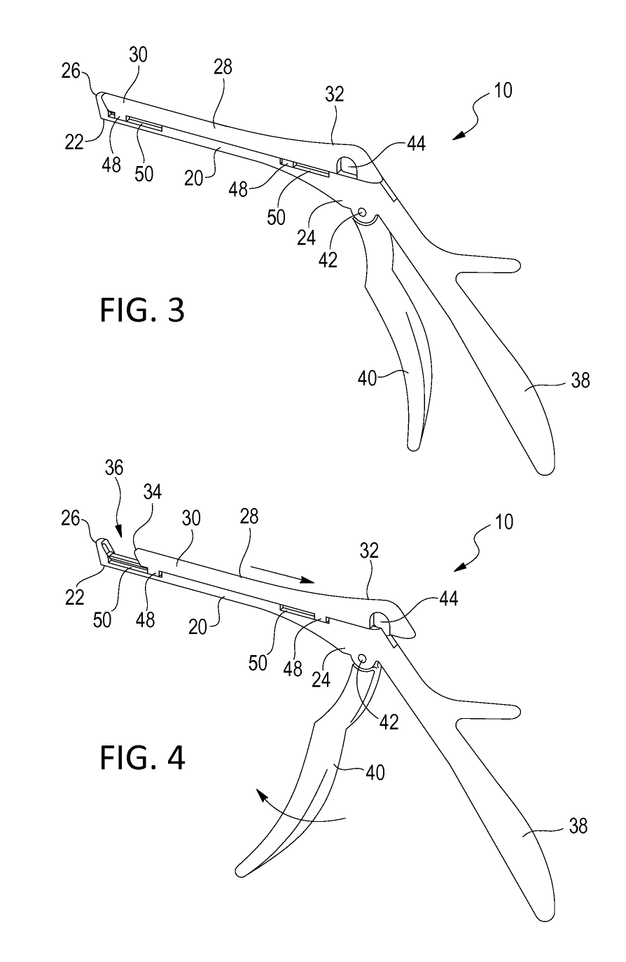

Referring to FIG. 3, by depressing second handle 40, which is biased against a spring member (not shown), cutting slide 28 is slid distally along shaft 20 until cutting edge 34 engages foot plate 26 of shaft 20. When this occurs, each tracking arm 48 is slid distally within a respective tracking slot 50. The interaction of the L-shaped tracking slots 50 with the L-shaped tracking arms 48 thereby allows only longitudinal movement of cutting slide 28 relative to shaft 20. No pivoting or vertical movement between shaft 20 and cutting slide 28 is allowed. Following cutting, second handle 40 is decompressed and rongeur 10 returns to the neutral position depicted in FIG. 1 with each tracking arm 48 being locked within a respective tracking slot 50.

When rongeur 10 requires cleaning it is necessary to gain access to the spaces formed between cutting slide 28 and shaft 28 where blood and tissue tend to accumulate. This is accomplished by first positioning tracking arms 48 within tracking slots 50 in a manner that allows tracking arms 48 to be disengaged from tracking slots 50. Referring to FIGS. 4 through 8, to disengage tracking arms 48 from tracking slots 50, second handle 40 to pivoted distally thereby sliding cutting slide 28 proximally until each tracking arm 48 contacts the proximal end wall of its respective tracking slot 50. In this position, horizontal portion 56 of each tracking arm 48 can be lifted or pivoted vertically out of its respective tracking slot 50 since overhang 54 extends longitudinally from the distal end wall of each slot 50 end wall but stops short of contacting the proximal end wall. This creates a space between the proximal edge of overhang 54 and the proximal end wall of each slot 50 through which a respective tracking arm 48 can be removed vertically from its tracking slot 50.

Upon removal of tracking arms 48 from tracking slot 50, cutting slide 28 is pivoted along a horizontal axis defined by the hinge assembly off to a lateral side of rongeur 10 away from the tracking slot 50 in proximal end 24 of shaft 20. Further, head 44 of second handle 40 is removed from open notch 46. The hinge assembly is positioned within an open gap 60 in a lateral side of shaft 20 opposite to tracking slot 50 located in proximal end 24 of shaft 20. Gap 60 includes a distal end wall 62, a proximal end wall and a pin 64 extending therebetween and in parallel to a length of shaft 20. Pin 64 defines the axis about which cutting slide 28 pivots. Cutting slide 28 is coupled to pin 64 by a cylindrical barrel 66 that extends downwardly from a lower surface of cutting shaft 28. Barrel 66 has an inner circumference large enough to allow barrel 66 to rotate about pin 64 when it is desired to partially disengage cutting slide 28 from shaft 20 and slide longitudinally along pin 64 when rongeur 10 is in use. In this manner, the hinge assembly allows both pivoting and sliding of cutting slide 28 relative to shaft 20.

Rongeur 12 is depicted in FIGS. 9 through 14. Rongeur 12 includes a hinge assembly having a pivot axis that extends parallel to the length and along a central axis of shaft 20. To accommodate a pivot axis along the central axis of shaft 20, shaft 20 includes an open cavity 68 formed in an inner surface 70 of shaft 20 along the central axis thereof within interior wall 52 between tracking slots 50. Extending upwardly from inner surface 70 is a pair of loops 72 with one loop located immediately adjacent to the distal end of cavity 68 and another loop located immediately adjacent to the proximal end of cavity 68. Loops 72 are arranged to receive there between a T-shaped sliding member 74 having an elongate opening 76 aligned with loops 72. T-shaped sliding member 74 is slidably supported by cutting slide which includes a T-shaped slot 77 for inserting T-shape sliding member 74 through cutting slide 28 and into cavity 68. Sliding member 74 is received within cavity 68 and held in place between loops 72 by a locking pin 75 that is inserted through loop 72 on the proximal end of cavity 68, through elongate opening 76 in sliding member 74 and finally through loop 72 in the distal end of cavity 68.

In use, when second handle 40 is compressed, cutting slide 28 slides along shaft 20 toward foot plate 26. During the movement of cutting slide 28, T-shaped sliding member 74 remains stationary while T-shaped slot 77 slides along sliding member 74. To allow for a sufficient sliding distance for cutting slide 28, T-shaped slot 77 has a length that is as least as long as the length of breach 36.

When it is desired to clean rongeur 12, like rongeur 10, it is necessary to disengage tracking arms 48 from tracking slots 50 of the tracking assembly. The tracking assembly of rongeur 12 includes two pairs of corresponding tracking slots 50 and tracking arms 48 combinations, with a first pair being positioned about distal ends 22 and 30, as described above for rongeur 10, and a second pair being positioned about proximal ends 24 and 32. To disengage tracking arms 48 from their corresponding tracking slots 50, second handle 40 is pivoted distally thereby causing cutting slide 28 to slide proximally until tracking arms 48 contact the proximal end walls of tracking slots 50. In this arrangement, cutting slide 28 can be lifted vertically away from shaft 20 with the tracking arms being raised above inner surface 70 of shaft 20.

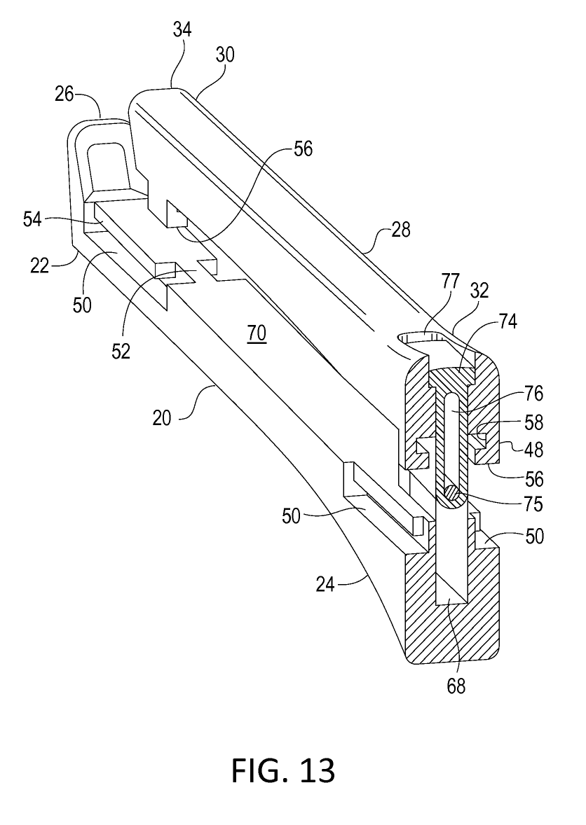

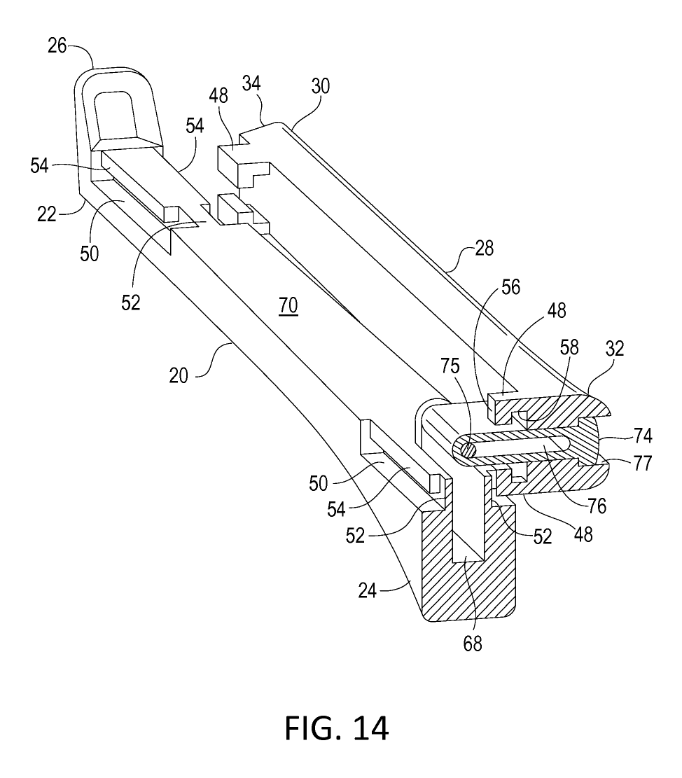

Referring to FIGS. 13 and 14, as cutting slide 70 is lifted vertically, sliding member 74 is lifted vertically by virtue of the interaction of a ledge 71 formed by the horizontal portion of T-shaped cutting slide 74 with a corresponding ledge 73 formed by the horizontal portion of T-shaped slot 77. As this occurs, sliding member 74 is removed from cavity 68 with the upward movement of cutting slide 28, the upward movement being limited by the interaction of pin 75 with the bottom most portion of elongate opening 76. Once the upward movement is stopped, sliding member 74 and cutting slide 28 can be pivoted about pin 75 either clockwise or counter-clockwise to expose inner surface 70 and the lower surface of cutting slide 28 to cleaning and repair.

Illustrated in FIGS. 15 through 33 are rongeur release mechanisms 100, 200 and 300 for preventing the inadvertent disengagement of tracking arms 48 from tracking slots 50 of the tracking assemblies of rongeurs 10 and 12 caused by the over expansion of second handle 40. In each embodiment, the release mechanisms operate by engaging a neck portion of second handle 40 between pivot point 42 and head 44 of second handle 40. Release mechanisms 100, 200 and 300 do so by providing the pathway along which the neck portion travels during operation of the rongeur with a section having an adjustable width that adjusts between a first width that is less than the width of the neck portion and a second width that is equal to or greater than the width of the neck portion. The adjustable width is adjusted from the first width to the second width by pressing the neck against one or more flexible or spring-biased members in the section with sufficient force to overcome and move the one or more biasing members, which define the width of the section. In certain instances, a blocking member is provided to ensure that the neck portion does not travel proximally a sufficient distance to allow disengagement of tracking arms 48 from tracking slots 50.

Referring to FIGS. 15 through 23, rongeur release mechanism 100 includes a pair of flexible metal strips 102, a blocking member 104 and a spring 106. Metal strips 102 are arranged adjacent to one another within a groove 108 formed in proximal end 24 of stationary shaft 20. Groove 108 extends longitudinally within stationary shaft 20 and partially defines a pathway 110 along which a neck 112 of second handle 40 travels when second handle 40 is depressed and extended. A pair of longitudinally extending, upwardly facing shelves 114 are formed within grove 108 and provide a surface upon which strips 102 are supported. The distal end of each strip 102 is fixed to an upright wall 116 of groove 108. Each strip 102 extends distally from the upright wall 116 and parallel to a lateral wall 118 of the groove 108 before bending inwardly towards the longitudinal axis of groove 108 and one another thereby fowling a chokepoint 120 having a width that is less than a width of neck 112. Each metal strip 102 then bends outwardly so that the proximal ends of strip 102 form a V-shape. The proximal ends of strips 102 are not fixed to groove 108.

In use, when it is desired to maintain tracking arms 48 seated within tracking slots 50, neck 112 is allowed to pivot and travel along pathway 110 with the neck's proximal movement being arrested by chokepoint 120. This occurs when the proximal side of neck 112 bears upon chokepoint 120 and strips 102 since chokepoint 120 has a width that is less than the width of neck 112. To ensure that neck 112 does not pass through chokepoint 120, blocking member 104 can be provided. Blocking member 104 includes a piston 122 slidably housed within a cylinder 124 formed within first handle 38. Spring 106 is provided in the bottom of cylinder 124 and biases piston upwardly and distally towards neck 112. When neck 112 is pivoted proximally and comes to bear on chokepoint 120, the proximal side of neck 112 also comes to bear upon piston 122. Piston 122 resists further movement of neck 112 due to the contact angle between neck 112 and piston 122 which transmits a majority of the force generated by pivoting neck 112 perpendicular to the axis of piston 122. A small amount of the force is transmitted axially along piston 122, but this amount is inadequate to overcome the compressive force of spring 106. By arresting proximal movement of neck 112, cutting slide 28 is prevented from sliding proximally a sufficient distance to disengage arms 48 from slots 50.

When it is desired to disengage tracking arms 48 from tracking slots 50, second handle 40 is pressed away from first handle 38 with sufficient force to cause neck 112 to bear against strips 102 and spread strips 102 apart such that chokepoint 120 has a width that is equal to or greater than the width of neck 112. When this occurs, neck 112 is able to move proximally beyond chokepoint 120 thereby allowing cutting slide 28 to slide proximally a sufficient distance to allow disengagement of arms 48 from slots 50. In those embodiments that include blocking member 106, piston 122 must be slid downwardly into cylinder 124 of first handle 38 before second handle 40 is moved away from first handle 38. Piston 122 is moved downwardly by manually pressing on a thumb trigger 126 that slidably rests on an outer surface of second handle 38. Thumb trigger 126 is coupled to piston 122 by a connecting portion 128 that extends there between and through a slot 130 in second handle 38.

Referring to FIGS. 24 through 29, rongeur release mechanism 200 includes a pair of bearing assemblies, each including a ball bearing 202, a spring 204 and a housing 206. The bearing assemblies are arranged opposite one another within a groove 208 formed in proximal end 24 of stationary shaft 20. Groove 208 extends longitudinally within stationary shaft 20 and partially defines a pathway along which a neck 212 of second handle 40 travels when second handle 40 is depressed and extended. Each housing 206 extends radially from a longitudinal axis of groove 208 through a lateral wall 214 of groove 208. Housings 206 each form a cylinder having an end wall 216 and a continuous sidewall 218. Spring 204 and bearing 202 are located within the cylinder with spring 204 being biased against end wall 216 thereby forcing bearing 202 radially inward toward the opposite bearing 202. Inward radial movement of each bearing 202 is prevented by an inner housing wall 219 having an opening with a diameter that is less than the diameter of bearing 202. This allows bearings 202 to partially protrude into groove 208 while maintaining the bearings within housing 206. With bearings 202 forced towards one another, a chokepoint 220 is formed there between. The chokepoint may have a width of zero to more than 0.25 inch.

In use, when it is desired to maintain tracking arms 48 seated within tracking slots 50, neck 212 is allowed to pivot and travel along the pathway with the neck's proximal movement being arrested by chokepoint 220. This occurs when the proximal side of neck 212 bears upon chokepoint 220 and bearings 202 since chokepoint 220 has a width that is less than the width of neck 212. By arresting proximal movement of neck 212, cutting slide 28 is prevented from sliding proximally a sufficient distance to disengage arms 48 from slots 50.

When it is desired to disengage tracking arms 48 from tracking slots 50, second handle 40 is pressed away from first handle 38 with sufficient force to cause neck 212 to bear against bearings 202 and depress bearings 202 and springs 204 such that chokepoint 220 has a width that is equal to or greater than the width of neck 212. When this occurs, neck 212 is able to move proximally beyond chokepoint 220 thereby allowing cutting slide 28 to slide proximally a sufficient distance to allow disengagement of arms 48 from slots 50.

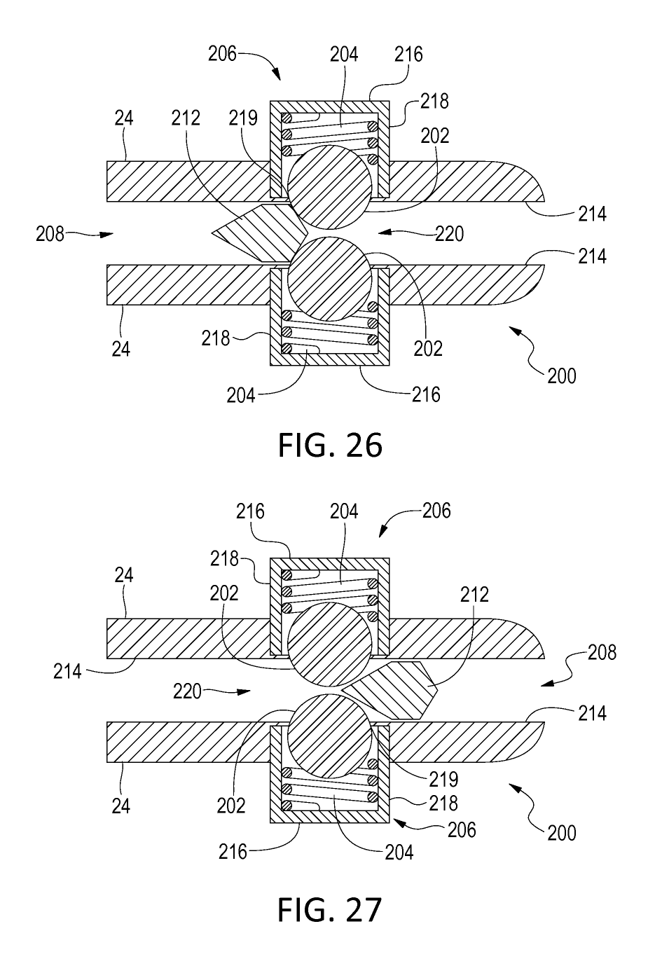

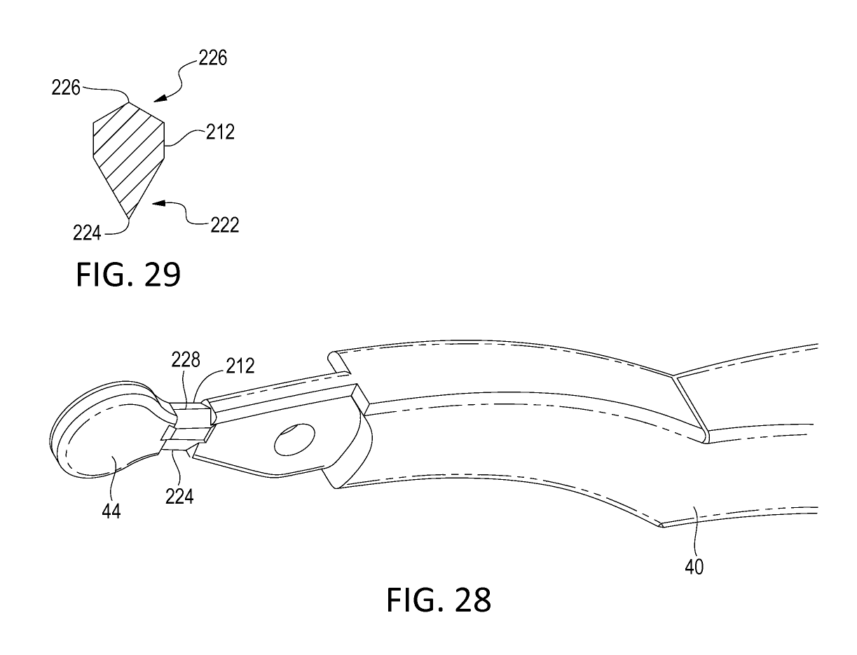

Referring to FIGS. 26 through 29, to increase the amount of force required to depress bearings 202 and springs 204 when disengaging aims 48 from slots 50, while decreasing the amount of force required to re-engage arms 48 with slots 50, neck 212 of second handle 40 may include a proximal side having a substantially blunt profile while the distal side has a pointed profile. Specifically, referring to FIGS. 28 and 29, the distal side of neck 212 may include a V-shaped profile 222 including an acute angle and terminating in a point 224 which allows neck 212 to more easily spread bearings 202 apart thereby allowing neck 212 to pass through chokepoint 220. The proximal side of neck 212, on the other hand, may include a broad, flat or blunt profile 226 including a point 228 forming an obtuse angle.

Referring to FIGS. 30 through 33, rongeur release mechanism 300 includes a pair of flexible metal strips 302, a blocking member 304 and a spring 306. Metal strips 302 are arranged adjacent to one another within a groove 308 formed in proximal end 24 of stationary shaft 20. Groove 308 extends longitudinally within stationary shaft 20 and partially defines a pathway along which a neck 312 of second handle 40 travels when second handle 40 is depressed and extended. A pair of longitudinally extending, upwardly facing shelves 314 are formed within grove 308 and provide a surface upon which strips 302 are supported. The distal end of each strip 302 is fixed to an upright wall 316 within groove 308. Each strip 302 extends distally from the upright wall 316 and parallel to a lateral wall 318 of the groove 308 before bending inwardly towards the longitudinal axis of groove 308 and one another thereby forming a chokepoint 320 having a width that is less than a width of neck 312. Each metal strip 302 then bends outwardly so that the proximal ends of strip 302 form a V-shape. The proximal ends of strips 1302 are not attached to groove 308.

In use, when it is desired to maintain tracking arms 48 seated within tracking slots 50, neck 312 is allowed to pivot and travel along pathway 310 with the neck's proximal movement being arrested by chokepoint 320. This occurs when the proximal side of neck 312 bears upon chokepoint 320 and strips 302 since chokepoint 320 has a width that is less than the width of neck 312. To ensure that neck 312 does not pass through chokepoint 320, blocking member 304 can be provided. Blocking member 304 includes a piston 322 slidably extending through a sidewall of stationary shaft 20, into and across groove 308 and terminating in a blocking portion 324 having a greater diameter than piston 322. The outer end of piston 322 terminates in a button 326. A spring 328 is provided about the piston 322 between button 326 and the outer surface of stationary shaft 20. In this way, piston 322 is biased against the outer surface of stationary shaft 20 so that blocking portion 324 is maintained within pathway 110 during operation of the rongeur. When neck 312 is pivoted proximally and comes to bear on chokepoint 320, the proximal side of neck 312 also comes to bear upon blocking portion 324. By arresting proximal movement of neck 312, cutting slide 28 is prevented from sliding proximally a sufficient distance to disengage arms 48 from slots 50.

When it is desired to disengage tracking arms 48 from tracking slots 50, second handle 40 is pressed away from first handle 38 with sufficient force to cause neck 312 to bear against strips 302 and spread strips 320 apart such that chokepoint 320 has a width that is equal to or greater than the width of neck 312. However, before neck 312 can travel proximally beyond chokepoint 220, blocking portion 324 must be pressed into an opening within lateral wall 318 by pressing button 326 and depressing spring 328 thereby pushing piston 322 and blocking portion 324 laterally with blocking portion 324 being received within lateral wall 318. When this occurs, pathway 310 is no longer blocked by blocking portion 324. While piston 322 continues to extend across pathway 310, the difference between the diameters of blocking portion 324 and piston 322 is great enough to allow sufficient additional proximal movement of cutting slide 28 to provide disengagement of tracking arms 48 from tracking slots 50.

As will be apparent to one skilled in the art, various modifications can be made within the scope of the aforesaid description. Such modifications being within the ability of one skilled in the art form a part of the present invention and are embraced by the claims below.

It is claimed:

1. A surgical rongeur comprising,
a shaft member including a foot plate and a handle,
a cutting slide coupled to the shaft member, the cutting slide including a cutting edge that is arranged to engage the foot plate,
a trigger pivotably coupled to the shaft member, the trigger including a neck portion that extends through the shaft member and a hammer portion that is received within a niche in the cutting slide, and
a pathway within the shaft member along which the neck is arranged to travel when the trigger is depressed and extended,
wherein the pathway includes a section having an adjustable width that is selectively adjustable between a first width that is less than a width of the neck and a second width that is greater than or equal to the width of the neck.

2. The surgical rongeur according to claim 1 wherein the section includes a flexible strip having a free end and another end anchored to the shaft member.

3. The surgical rongeur according to claim 1 wherein the section includes at least one flexible strip.

4. The surgical rongeur according to claim 1 wherein the section includes a first flexible strip arranged along a first lateral side of the section and a second flexible strip arranged along a second side lateral of the section.

5. The surgical rongeur according to claim 1 wherein the section includes at least one member biased towards a lateral side of the section.

6. The surgical rongeur according to claim 1 wherein the section includes a pair of bearings biased against opposing sides of the section.

7. The surgical rongeur according to claim 1 wherein the section includes at least one spring biased bearing.

8. The surgical rongeur according to claim 1 wherein a proximal end section of the pathway is selectively blocked by a biased member.

9. The surgical rongeur according to claim 1 further comprising a rod member biased toward the neck and extending into a proximal end of the pathway.

10. The surgical rongeur according to claim 1 further comprising a finger actuated protrusion slidably coupled to the handle and a rod member that is biased toward the neck.

11. The surgical rongeur according to claim 1 further comprising a manually actuatable stop extending into a proximal end of the pathway.

12. A surgical rongeur comprising,
a bottom shaft having a foot plate,
a top shaft slidably coupled to the bottom shaft, the top shaft having a cutting edge, and
a trigger assembly including a trigger having a neck portion that extends through a pathway in the bottom shaft and an upper end that is engaged with the top shaft, the pathway including a section having an adjustable width.

13. The surgical rongeur according to claim 12 wherein the adjustable width is adjustable between a first width that is less than a width of the neck and a second width that is greater than or equal to the width of the neck.

14. The surgical rongeur according to claim 12 wherein the section includes a first flexible metal strip arranged to a first side of the neck and a second flexible metal strip arranged to a second side of the neck.

15. The surgical rongeur according to claim 12 wherein the section includes a pair of opposing biased members.

16. The surgical rongeur according to claim 12 further comprising a slidable member extending below the top shaft and bearing against a proximal side of the neck.

17. The surgical rongeur according to claim 12 wherein the trigger assembly further includes a slidable member extending from a handle into the pathway.

18. A surgical rongeur comprising,
a top shaft slidably coupled to bottom shaft,
a trigger assembly having a trigger that extends upwards through a pathway in the bottom shaft and engages the top shaft, and
a section of the pathway having an adjustable width.

19. The surgical rongeur according to claim 18 wherein the adjustable width includes a first width arranged to arrest movement of the trigger along the passageway and a second width that is arranged to allow movement of trigger along the passageway.

20. The surgical rongeur according to claim 18 wherein the adjustable width is defined between moveable metal pieces.

21. The surgical rongeur according to claim 18 wherein the adjustable width is defined between flexible members.

22. The surgical rongeur according to claim 18 wherein the adjustable width is defined between spring-biased members.

23. The surgical rongeur according to claim 18 wherein the adjustable width is partially defined by a biased member.

24. The surgical rongeur according to claim 18 wherein the trigger assembly includes a finger-actuated blocking member arranged to arrest movement of the trigger within the pathway.

25. The surgical rongeur according to claim 24 wherein the blocking member includes a rigid portion that extends into the pathway and a flexible portion that extends into a handle portion of the bottom shaft, the flexible portion bearing against the rigid portion.

26. The surgical rongeur according to claim 18 wherein the adjustable width ranges between 0 and 0.25 inch.
